(19) United States
(12) Patent Application Publication
(10) Pub. No.: US 2024/0358389 A1
(43) Pub. Date: Oct. 31, 2024

(54) SURGICAL INSTRUMENT AND STEERING GEAR FOR SAME

(71) Applicant: KARL STORZ SE & CO. KG, Tuttlingen (DE)

(72) Inventor: Sven Axel GRÜNER, Trossingen (DE)

(73) Assignee: KARL STORZ SE & CO. KG, Tuttlingen (DE)

(21) Appl. No.: 18/291,770

(22) PCT Filed: Jul. 25, 2022
(86) PCT No.: PCT/EP2022/070834

§ 371 Date: Jan. 24, 2024

(30) Foreign Application Priority Data
Jul. 28, 2021 (DE) 10 2021 119 536.5

(51) Int. Cl. A61B 17/29; A61B 17/00; A61B 34/00; A61B 34/30

(52) U.S. Cl. CPC A61B 17/29; A61B 34/30; A61B 34/70; A61B 2017/00398; A61B 2017/00477; A61B 2017/0069; A61B 2017/00858; A61B 2017/292; A61B 2017/2927

(57) ABSTRACT

A steering gear for a surgical instrument includes two motorized drives and spatially orients a swash plate by way of the adjustment angles of the two drives, in order to control a deflection mechanism of the surgical instrument. The swash plate is arranged in a steering ring and each of the two motorized drives has a drive shaft driven by a motor and are operatively connected with the steering ring via a respective force transmitter. The first force transmitter contacts the steering ring on a first active portion, and the second force transmitter contacts the steering ring on a second active portion, wherein the steering ring is mounted cardanically around a gimbal center, wherein the active portions have a common sphere center at which a first tilt axis and a second tilt axis is defined by a contact point at a respective first transmitter and second transmitter.

### CROSS-REFERENCE TO RELATED APPLICATIONS

This application is the U.S. national stage of PCT/EP2022/070834 filed on Jul. 25, 2022, which claims priority of German Patent Application No. 10 2021 119 536.5 filed on Jul. 28, 2021, the contents of which are incorporated herein.

### TECHNICAL FIELD

The disclosure relates to a steering gear of a surgical instrument for bending a tool tip at an angle by means of a spatially orientable swash plate, and a surgical instrument which comprises such a steering gear.

### BACKGROUND

From the prior art, surgical instruments are known which can be guided manually or by a robot and and which have tools whose tool tip can be pivoted by means of a plurality of pivot members engaging in one another. These pivot members are connected to a multiplicity of steering wires or steering cables in order to achieve delicate control of the tool tip. With a large number of thin steering wires, compared to a few thicker steering wires, it is possible to achieve a more uniform force distribution in all deflection directions.

It is known from U.S. Pat. No. 5,454,827 B2 to couple such steering wires to a spatially adjustable plate arranged proximally in an actuation unit, which plate is connected via a rod to a manually actuated control lever, such that a movement of the spatially adjustable swash plate causes a corresponding relative movement of the distal-side pivot members and thus a pivoting of the tool tip.

The design of the drive for the steering wires with the spatially adjustable swash plate, on which all four steering wires are mounted, is advantageous in that this enables a spatially compact structure and only requires the movement of one component in order to address all the steering wires. This design means that it is possible to use just a small number of steering wires, and the spatially adjustable plate, which serves as the drive for the steering wires, can be manually operated, both of which factors affect the sensitivity and reproducibility of the adjustment of the distal-side pivot members.

U.S. Pat. No. 7,699,855 B2 discloses a surgical instrument which has an interface so as to be able to connect the instrument to a robot arm. All the drives that control the instrument are arranged in the robot arm. The rotation angles of the drives are transmitted to the instrument via clutch disks in a common separation plane.

It is also known, in a surgical instrument with a compact steering gear, to transmit the adjustment angles of two drives directly to the swash plate in order to orient the latter for controlling the tool tip. To do this, steering wires are fastened to the swash plate so that the tip of the tool can be steplessly and smoothly controlled by alignment of the swash plate. For this purpose, the known steering gear has two drive bevel gears offset by 180° from each other, which are arranged on a common axis of rotation that runs perpendicular to an instrument longitudinal axis, each with an associated motor. The swash plate is arranged between the drive bevel gears and is mounted in a steering ring which is connected for conjoint rotation to a third bevel gear which engages with the two drive bevel gears and is rotatable about an axis of rotation perpendicular to the instrument longitudinal axis and perpendicular to the common axis of rotation of the drive bevel gears. The gear chain is supplemented by a fourth bevel gear, which is arranged on the axis of rotation of the third bevel gear, offset by 180° from the third bevel gear, and is in engagement with the two drive bevel gears, wherein the steering ring is mounted freely rotatably in the fourth bevel gear. The gear chain, closed in this way, ensures that all the bevel gears engage with one another and permits uniform force distribution.

The design of the drive for the steering wires with the spatially adjustable swash plate, on which all four steering wires are mounted, is advantageous in that this enables a spatially compact structure and only requires the movement of one component in order to address all the steering wires.

WO 2014/004242 also describes such an interface, wherein the drives are installed in the robot arm. The above design is associated with a complex structure and with an indirect control. The drives are not directly arranged in the surgical instrument, which means that the swash plate is not controlled linearly.

U.S. Pat. No. 10,105,128 B2 also discloses a control of such a tool tip, this control being effected by way of a mechanism comprising toothed disk segments and articulated rods for transmitting the movement of the drives to the swash plate.

### SUMMARY

Proceeding from this prior art, it is an object of the present disclosure to provide alternative control and guidance of the steering ring.

This object is achieved by a steering gear having the features of claim 1.

The further object of making available a surgical instrument with alternative guidance of the steering ring is achieved by the surgical instrument having the features of independent claim 9.

Developments of the steering gear and of the surgical instrument are set forth in the respective dependent claims.

A first embodiment of the steering gear is designed for a surgical instrument which can be arranged at the proximal end of a shaft that defines a longitudinal axis B and has a deflection mechanism at the distal end. The steering gear has two motorized drives and is designed to spatially orient a swash plate via the adjustment angles of the two drives. The swash plate is further designed to control the distal deflection mechanism of the surgical instrument.

According to the disclosure, the swash plate is arranged in a steering ring. The first of the two motorized drives has a first drive shaft, which is driven by a first motor and which is directly in operative connection with the steering ring via a first force transmitter, since the first force transmitter directly contacts the steering ring at a first active portion. For this purpose, the first force transmitter is arranged on the first drive shaft, which defines a first drive axis C. Furthermore, the second of the two motorized drives has a second drive shaft, which is driven by a second motor and which is directly in operative connection with the steering ring via a second force transmitter, since the second force transmitter directly contacts the steering ring at a second active portion. The second force transmitter is arranged on the second drive shaft, which defines a second drive axis C′. The steering ring is mounted cardanically around a gimbal center, such that a guided movement is enabled in two spatial axes, whereby the tool tip can be controlled in a targeted manner. To ensure that the engagement with the force transmitters is also maintained in the event of the steering ring tilting, the steering ring has a spherical or spherical segment shape at least at the first and second active portions around the gimbal center. The spherical segment-shaped active portions have a common sphere center point, which corresponds to the gimbal center. In this way, a first tilt axis E, which is defined by a contact point of the first force transmitter with the steering ring and by the gimbal center, and a second tilt axis E′, which is defined by a contact point of the second force transmitter with the steering ring and by the gimbal center, intersect at right angles in the gimbal center. This means that the drives touch the steering ring with the active portions, in such a way that the normals, defined as tilt axes E, E′, of the surfaces touching each other at the respective contact points intersect vertically in the gimbal center.

“Spherical segment-shaped” is understood as meaning shapes that can be defined as a part of a spherical body which is separated by a plane cut and whose curved surface is designated as a spherical cap. In the present case, “spherical segment-shaped” is also intended to include spherical disk shapes which arise as part of a spherical body in which two sphere segments (“polar caps”) have been removed by parallel cutting planes, wherein the curved surface of the spherical disk is designated as a spherical zone. For example, the steering ring can have a spherical disk shape in which the two parallel cutting planes are equidistant or symmetrical to the diameter plane, such that the steering ring surface is formed as a spherical zone symmetrical to the diameter plane.

At least in the region of the active portions, the steering ring thus has a spherical or partial spherical character, wherein a circle center of a sphere, which is defined by the spherical or partial spherical active portions, is the gimbal center. In principle, it is sufficient if the surface of the steering ring is spherical only at the two active portions that engage with the force transmitters.

This basic principle is common to all the variants of the force transmitter described below: At the contact points between force transmitter and steering ring, the movement of the force transmitter, when viewed infinitesimally, is always parallel to the main axis B of the surgical instrument at this point and directed forward or rearward in relation to the main axis B, depending on the direction of movement of the force transmitter. This movement is transmitted to the steering ring. The gimbal suspension of the steering ring causes the latter to tilt about one or both tilt axes E, E′.

Here, “force transmitter” designates any component that directly receives the movement initiated by motors, whether rotational or linear, and can pass it on to the active portion of the steering ring.

Here, “active portion” is the region of the steering ring that can enter into a force-transmitting operative connection with the force transmitter, i.e. is directly in contact with the force transmitter, e.g. by means of a frictional operative connection using friction elements, or that is engaged therewith, e.g. by means of a toothing or other suitable force-transmitting operative connections.

Advantageously, the steering ring, which comprises the swash plate, can be directly controlled from the drive motors by means of the force transmitters. No further deflection mechanisms or gear transmissions are necessary, and therefore the shortest possible transmission chain is permitted. Such a direct and short force transmission is advantageously associated with low play and with a reduction in transmission and friction losses, and, on account of the linear transmission behavior, it also permits simple software control in order to precisely control the component that is to be controlled.

In another embodiment of the steering gear according to the disclosure, the cardanic mounting of the steering ring is provided by the fact that
a) the steering ring is suspended cardanically on a first fastening device. The fastening device is a bracket arranged on a portion of the steering ring facing away from the active portions. Furthermore, at both ends, the bracket is mounted on the housing pivotably about a pivot axis A which runs perpendicular to the longitudinal axis B and perpendicular to the drive axes C, C′. The bracket has a receiving opening at its center, wherein the steering ring is mounted in the receiving opening rotatably about an axis of rotation D perpendicular to the pivot axis A. The swash plate can preferably be mounted rotatably in the steering ring. in order to also permit a rotational movement of the swash plate in the steering ring, but it can optionally also be firmly connected to the steering ring or be in one piece with the steering ring, resulting in a combined component of steering ring and swash plate.

The rotatable mounting of the bracket on the housing comprises bearing pins, by means of which the bracket is rotatably mounted about the pivot axis A on both sides in the housing, such that the steering ring, which can rotate exclusively about its transverse axis and vertical axis, including also superpositions, is defined in its position in space, and a rotation about the main axis B is prevented. The above-mentioned gimbal suspension of the steering ring by means of a bracket is simple in design and can be precisely controlled. Moreover, by means of the bracket spanning it, the steering ring is additionally encased and protected.

As an alternative to the gimbal suspension by means of a bracket, the cardanic mounting of the steering ring can be provided by the fact that
b) the steering ring is mounted cardanically on a main shaft, extending coaxially to the longitudinal axis B of the shaft, via the swash plate, which is mounted rotatably in the steering ring about the longitudinal axis B. For this purpose, the swash plate is mounted pivotably on a universal joint plate via two bearing pins offset by 180° from each other, i.e. diametrically and coaxially arranged bearing pins, and the universal joint plate is in turn mounted pivotably on the main shaft via two bearing pins offset by 180° from each other, i.e. diametrically and coaxially arranged bearing pins, wherein the pairs of bearing pins of the swash plate and of the universal joint plate are offset by 90° from each other.

To ensure that the steering ring here can also rotate exclusively about the transverse axis and vertical axis, including superpositions, and that its position in space is fixed and a rotation about the main axis B is prevented, the steering ring can optionally be coupled to a housing in a rotationally fixed manner with respect to the longitudinal axis B, for example by means of a pin which extends radially outward from the steering ring, for example from the underside of the steering ring between the active portions, and is guided in a groove of the housing, which groove extends parallel to the longitudinal axis B along the radius of movement of the pin.

If the first drive shaft is moved by the first motor and thus the first force transmitter is rotated about the drive axis C, this movement is transmitted to the steering ring at the first active portion by virtue of the operative connection between the steering ring and the first force transmitter. If the second force transmitter meanwhile remains stationary, the steering ring is moved at its contact point with the first force transmitter and remains at the contact point of the second active portion with the second force transmitter, as a result of which the steering ring is tilted, specifically about the second tilt axis E′, which passes through the fixed contact point of the second active portion with the second force transmitter and lies at an angle of 45° both to the axis of rotation D and the axis of rotation A. Conversely, a rotation of the second force transmitter about the drive axis C′ causes the steering ring to rotate about the first tilt axis E which, analogously to the second tilt axis E′, lies at an angle of 45° both to the axis of rotation D and the axis of rotation A, and which this time passes through the fixed contact point of the first force transmitter and the first active portion of the steering ring. When both force transmitters rotate, the tilting movements are superposed and the steering ring, and thus the swash plate, can be tilted in space about both tilt axes E, E′, i.e. two-dimensionally.

In another embodiment of the steering gear according to the disclosure, the operative connection provides an engagement, since the first and second force transmitters are rotationally mounted drive cones having a cone head comprising circumferentially a plurality of drive knobs forming a knob ring. In this case, the steering ring has recesses at the active portions, which recesses mesh with the drive knobs of the two knob rings. The cone head preferably has a partially or fully toroidal shape, in order to achieve a good contact point to the steering ring.

Preferably, it can be provided that the knobs in a further embodiment of the steering gear according to the disclosure have a hemispherical or a semi-ellipsoid shape. These shapes permit safe meshing with the active portion of the steering ring and also good force transmission from the respective force transmitter to the steering ring. Corresponding to the shape of the drive knobs, the recesses are either concentrically arranged annular grooves or arc-shaped grooves, which form as it were a hypoid-like gear. The rings are best meshed by hemispherical knobs and the arc-shaped grooves are best meshed by semi-ellipsoid knobs. In both cases, the force is transmitted without jolts and with low friction. Through the engagement of the knobs in the recesses, considerable drive forces can be transmitted precisely and without occurrence of slipping.

In an alternative embodiment of the steering gear according to the disclosure, the first and second force transmitters can be worm shafts which run parallel to the longitudinal axis B. For this purpose, the steering ring has, on its active portions, concentrically arranged recesses/annular grooves, which mesh with the respective worm shaft.

The two aforementioned embodiments provide form-fit operative connections in which the two components, steering ring and force transmitter, engage with each other in the manner of a toothing. This permits good and precise force transmission, which then permits precise control of the swash plate.

In a further alternative embodiment of the steering gear according to the disclosure, the operative connection is provided by friction. This is achieved by the fact that the first and second force transmitters are rotationally mounted drive cones having a cone head, which comprises circumferentially a friction element. The friction element is preferably a material which is applied around the cone head and which, with the active portion of the steering ring. provides a higher friction than the material from which the cone head is made. For a better hold on the cone head, such a friction-increasing material can also be introduced into a groove surrounding the cone head. Alternatively, the friction element can be a friction band which is applied circumferentially around the cone head and which is a rubber band or a band with a roughened surface or a band with teeth or micro-serrations or knobs.

Here, “friction” denotes an operative connection that provides frictional engagement, wherein the frictional force between two components that come into contact (here the steering ring and the drive cone) is so great that, when the drive cone is rotated, it as it were rolls on the steering ring and thus sets the latter in motion. In this context, “friction element” denotes any component that provides a required material property, such as an increased frictional resistance (e.g. sliding friction force). This can be achieved through a certain choice of material or surface property, such as the abovementioned roughening or toothing. The risk of slippage, which cannot necessarily be completely excluded in the case of a frictional connection, can be reduced by an increased contact pressure, which is determined by the positioning of the force transmitters in relation to the steering ring.

The advantage of this variant is that the frictional operative connection permits an almost continuous tilting of the steering ring. i.e. steplessly. This also permits precise control of the swash plate. Other advantages of this variant are an absolute freedom from play and also low-cost components or production.

In order to combine the respective advantages of the positive and frictional operative connections, or to minimize the respective disadvantages, mixed forms are also conceivable: In a further embodiment, it is therefore provided that the operative connection of the first force transmitter provides a form-fit engagement and the operative connection of the second force transmitter is provided by friction. In this case, the first force transmitter can be a rotationally mounted drive cone with a cone head, which circumferentially comprises a plurality of drive knobs forming a knob ring, or a worm shaft which runs parallel to the longitudinal axis B. On the first active portion, the steering ring accordingly has recesses which mesh with the drive knobs of the two knob rings or the worm shaft. The second force transmitter, which is likewise a rotationally mounted drive cone with a cone head, has a friction element circumferentially on the cone head, preferably a material which is applied around the cone head and which, with the second active portion of the steering ring, provides a higher friction than the material from which the cone head is made. Alternatively, the friction element can be a friction band which is applied circumferentially around the cone head and which is a rubber band or a band with a roughened surface or a band with teeth or knobs. Thus, by forming one drive cone with a positive toothing and the other one with friction, slippage and play are minimized. As a variant of this embodiment, a combined positive and frictional operative connection can also be realized together on a drive cone, for example by the latter having a knob ring which also has a frictional connection in addition to the positive fit provided by the toothing with the recesses on the active portion, by the surface of the drive cone with the knob ring or the surface of the active portion with the recesses being additionally provided with a rubber coating. thus preventing play. However, the underlying toothing contour ensures the form fit over the entire service life and prevents slippage while at the same time permitting high force transmission.

Moreover, in a further embodiment of the steering gear according to the disclosure, the rotationally mounted drive cones each have a cone shaft around which a bearing ring is arranged adjacent to the cone head, which bearing ring houses, for example, a roller bearing or a ball bearing. For this purpose, the housing has a base comprising a through-opening with two opening portions for each drive axle. The two opening portions of the through-openings are axially adjacent to each other, so that they merge into each other and form a shoulder, wherein they widen above the shoulder, facing the steering ring, to a diameter corresponding to the diameter of the bearing rings. This means that each through-opening has a first opening portion with a diameter corresponding to a diameter of the bearing ring, and a second opening portion, coaxial to the first opening portion, with a diameter smaller than the diameter of the first opening portion, such that the housing forms a shoulder in each through-opening. Each of the bearing rings then rests on the respective shoulder and is held securely. The drive shaft, which passes through the bearing ring and thus also both through-opening portions, is guided laterally and held in rotation. This allows the force applied by the motors to be transmitted directly to the drive bevel gears via the drive shafts.

In an alternative embodiment of the steering gear according to the disclosure, it is provided that the operative connection forms a linear drive, wherein the first and second force transmitters are toothed rods with a toothing portion. In this case, the steering ring has, on its active portion, concentrically arranged recesses which mesh with the toothed portions of the two toothed rods. In another alternative embodiment of the steering gear according to the disclosure, a cohesively bonded operative connection could also be achieved. For this purpose, tension means such as cords or bands could be fastened with both ends to the respective active portions of the steering ring and could be looped once around the respective force transmitter. These variants offer direct control of the swash plate in the case of narrow housings or in cases where motors have to be arranged differently for other constructional reasons.

In a first embodiment of a surgical instrument according to the disclosure comprising a shaft, an actuation unit arranged at the proximal end of the shaft, and a tool arranged at the distal end of the shaft with a tool tip which can be bent at an angle by means of a distal deflection mechanism and which can be controlled by a swash plate that can be spatially oriented by means of two drives, it is provided that the surgical instrument has a steering gear according to the disclosure for the spatial orientation of the swash plate.

By virtue of the steering gear according to the disclosure, the surgical instrument can be constructed in structurally simple and space-saving fashion, with the result that a simple connection to a robot arm can be enabled, in the case of which the movement of the drives can be transmitted directly to the tool tip. The result is a precisely controllable use of the surgical instrument.

In order to be able to adjust the spatially adjustable swash plate, a preferred embodiment of the surgical instrument can be provided in which the swash plate is mounted cardanically on a main shaft extending coaxially to a longitudinal axis B of the shaft. The steering ring is then mounted cardanically via the swash plate, which can also be referred to as an internal cardanic mounting. This is an alternative embodiment to an outer cardanic mounting of the steering ring via the bracket. This mounting may be advantageous depending on the design and available space of the surgical instrument.

In a further embodiment of the surgical instrument according to the disclosure, the actuation element is axially displaceably mounted in the shaft and is operatively connected to the actuation unit on the proximal side. The distal deflection mechanism of the bendable tool tip consists of pivot members which are arranged at the distal end of the shaft and are connected to the steering gear via the steering wires running in the longitudinal direction of the shaft. The steering wires are fastened to the swash plate, for example by means of a clamp connection, so that, in the case of damage, the steering wires can be easily replaced. This clamping connection can be effected, for example, by grub screws provided in radially introduced holes in the swash plate. However, the steering wires can also be less complex and therefore more cost-effective when fastened non-releasably to the swash plate, for example by welding or gluing, which may be preferred in single-use surgical instruments. Compared to known constructions, this construction is advantageous not only in that it is possible to use a small number of steering wires, specifically just four steering wires, and in that the spatially adjustable plate serving as a drive for the steering wires can be actuated exclusively manually, but also in that a plurality of steering wires can be chosen freely, thereby enabling sensitive and reproducible adjustment of the distal-side pivot members.

The surgical instrument according to the disclosure has the advantage that many thin steering wires can be used to control the pivotable tool tip and that, on account of the motorized drive for the spatially adjustable plate on which the steering wires are mounted proximally, this control is sensitive, precise and reproducible.

Further embodiments of the steering gear and of the surgical instrument, and some of the advantages associated with these and further embodiments, will be clearly and better understood from the following detailed description with reference to the accompanying figures. Objects or parts thereof which are substantially the same or similar may be provided with the same reference signs. The figures are merely a schematic illustration of an embodiment of the disclosure. The drawings, the description and the claims contain numerous features in combination. A person skilled in the art will advantageously also consider the features on an individual basis and combine them to form further advantageous combinations.

### BRIEF DESCRIPTION OF DRAWINGS

FIG. 1 shows a perspective view of the surgical instrument with a schematically illustrated actuation unit,

FIG. 2 shows a detailed perspective view of a first embodiment of the steering gear according to the disclosure without a force transmitter and with a part of a housing,

FIG. 3 shows a detailed perspective view of the first embodiment of the steering gear according to the disclosure with force transmitter and without housing,

FIG. 4 shows a sectional view of the steering gear according to the disclosure according to the first embodiment with force transmitter and housing,

FIG. 5 shows a detailed perspective view of another embodiment of the steering gear according to the disclosure with a rubber ring,

FIG. 6 shows a detailed perspective view of another embodiment of the steering gear according to the disclosure with drive heads with knobs,

FIG. 7 shows a detailed perspective view of yet another embodiment of the steering gear according to the disclosure with worm shafts,

FIG. 8 shows a detailed perspective view of yet another embodiment of the steering gear according to the disclosure for a linear drive with a toothed rod,

FIG. 9 shows a detailed view, according to FIG. 8, of the toothed rod,

FIG. 10 shows a perspective view of the actuation unit with a further embodiment of the steering gear according to the disclosure,

FIG. 11 shows a partial sectional view of the steering gear according to FIG. 10,

FIG. 12 shows a detailed view of a steering ring and a force transmitter according to FIGS. 10 and 11, and

FIG. 13 shows a sequence of steps in the production of the force transmitter according to FIG. 12.

### DETAILED DESCRIPTION OF PREFERRED EMBODIMENTS

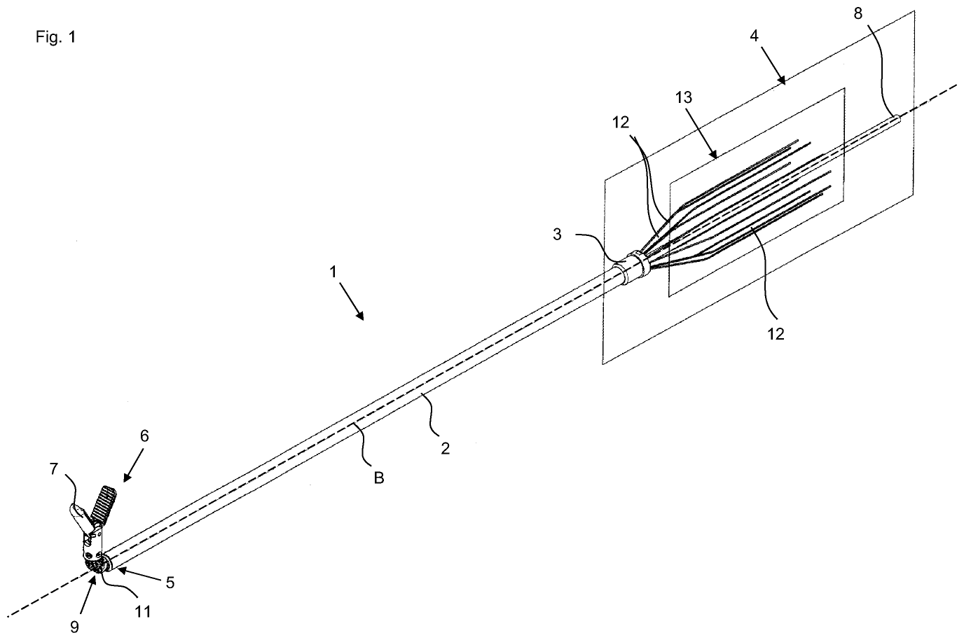

FIG. 1 shows schematically a surgical instrument 1 with a hollow shaft 2, an actuation unit 4 (indicated only schematically) arranged at the proximal end 3 of the shaft 2, and a tool tip 6, arranged at the distal end 5 of the shaft 2, with a tool 7. The tool 7 can be actuated via an actuation element 8 which is mounted axially displaceably in the shaft 2 and which is operatively connected at the proximal end to the actuation unit 4. The actuation unit 4 can be a manually actuated handle or a structural unit designed for robotic use, that is to say also actuated without manual intervention, which is advantageous for the reproducibility of the actuation. The tool 7 of the tool tip 6 can be, for example, a tool provided with jaw parts, as illustrated in FIG. 1, or an endoscope, an applicator or the like. The tool tip 6 is pivotable relative to the longitudinal axis B of the shaft 2 via a joint mechanism 9, wherein the joint mechanism 9 consists of pivot members 11 which are arranged at the distal end of the shaft 5 and which, by way of steering wires 12 extending in the longitudinal direction of the shaft 2, are connected to a steering gear 13, arranged at the proximal end 3 of the shaft 2, such that a movement of the proximal-side drive 13 causes a corresponding relative movement of the distal-side pivot members 11 and thus a pivoting of the tool tip 6. Even though exclusive use is made of the term steering wires 12 hereinabove and below, from a functional point of view use can also be made of steering cables, which is why the used term steering wires 12 should also be read and understood synonymously as steering cables.

In the illustrated embodiment, the actuation element 8, mounted axially displaceably in the shaft 2 and serving to actuate the tool consisting of two jaw parts for example, is designed as a pull/push rod.

The steering gear 13 for the steering wires 12 is formed in the surgical instrument 1 as a motorized steering gear 13.

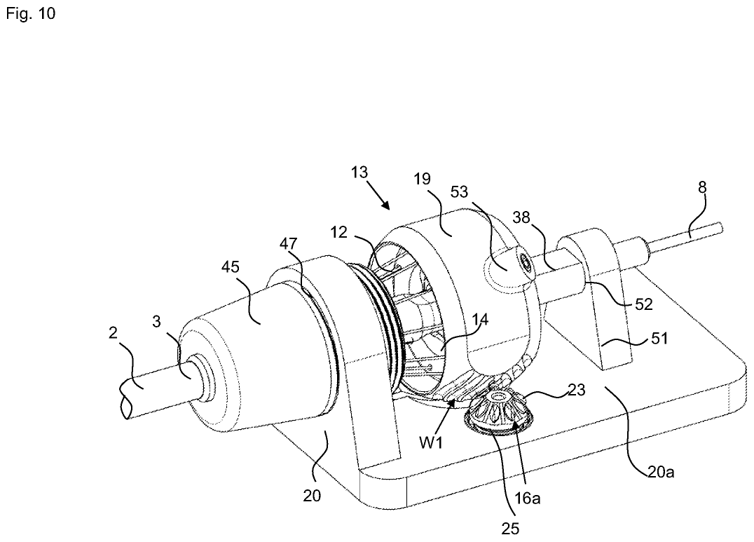

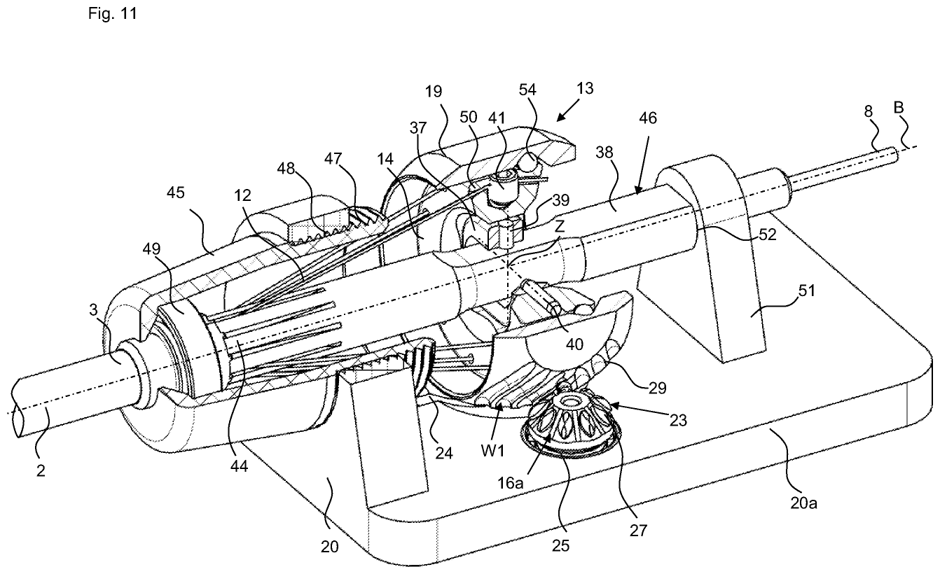

FIGS. 10 and 11 show an embodiment of the steering gear 13 with a spatially adjustable swash plate 14, in which embodiment the swash plate 14 is mounted cardanically on a main shaft 38 extending coaxially to the shaft 2, in order to move it relative to the longitudinal axis B of the shaft 2. In this case, the steering wires 12 are mounted on the swash plate 14 or fastened thereto such that, during the movement of the swash plate 14, the tool tip 6 is pivoted via the steering wires 12.

The number of steering wires 12 to be used for the motorized steering gear 13 there is ten, but a steering gear 13 according to the disclosure is not limited to this. The steering wires 12 of the swash plate 14, which extend parallel to the longitudinal axis B of the shaft 2 at the distal side, emerge from the shaft 2 via a guide element 44 and are fed to the swash plate 14, with the diameter of the steering wire bundle increasing. For fixing the steering wires 12, the swash plate 14 has through-holes 50 formed in it for each steering wire 12, which are fixed via grub screws 41 introduced radially into the swash plate 14. A radial arrangement of the grub screws 41 requires that the steering wires 12 are mounted on the swash plate 14 before the swash plate 14 is arranged in the steering ring 19. Therefore, in contrast to what is shown, the grub screws can be arranged slightly inclined or displaced in a deviation from the radial direction, such that they remain accessible despite the arrangement of the swash plate in the steering ring.

Alternative ways of fastening the steering wires to the swash plate are conceivable; for example, the steering wires can be connected with force-fit engagement to a clamping plate arranged proximally behind the swash plate, or simply non-releasably by welding or gluing (not shown in the figures).

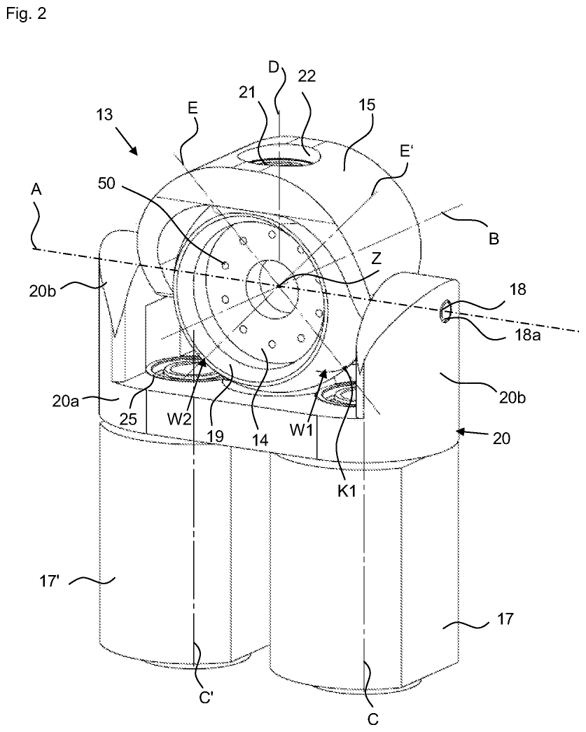

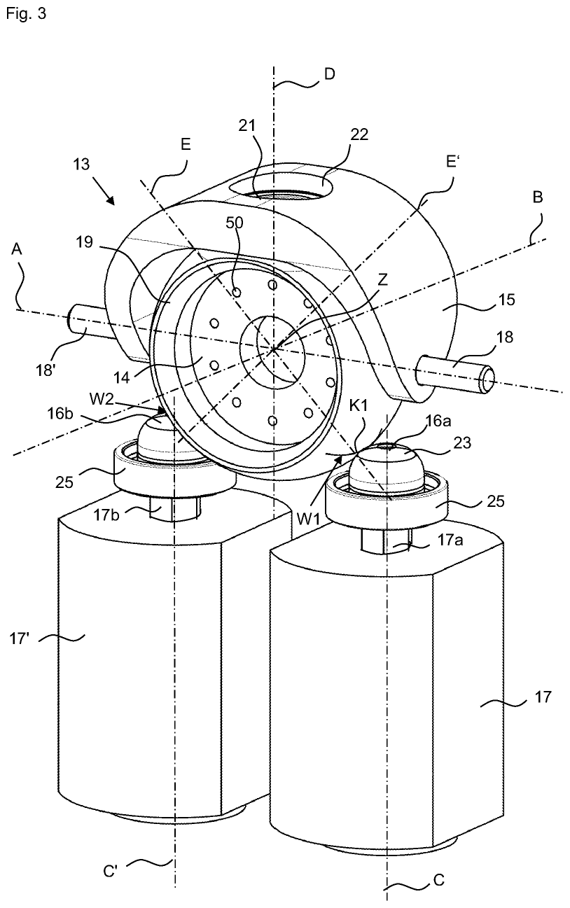

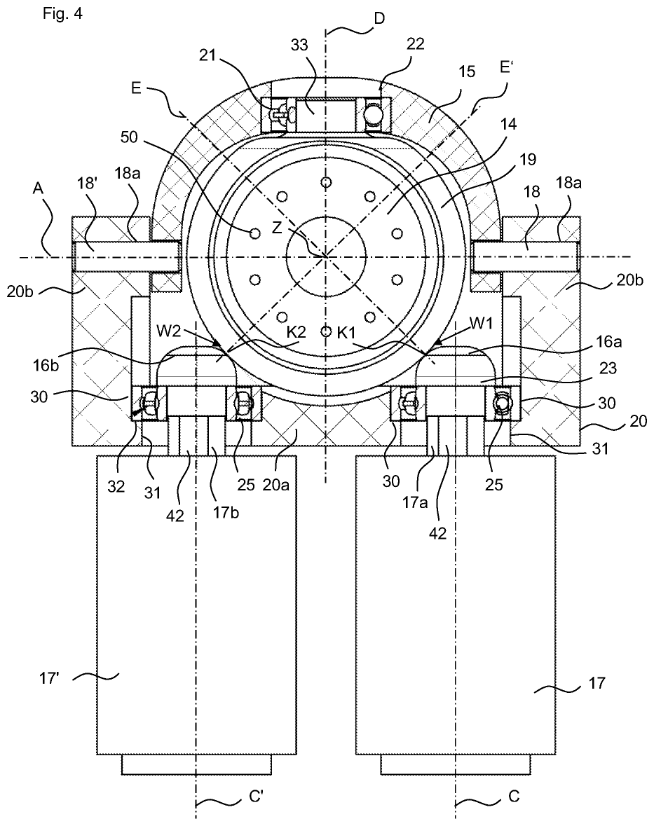

In FIG. 2, the central components of the steering gear 13, which are also the basis for the further embodiments, in particular for the embodiments shown in FIGS. 3 to 8, are shown in a simplified form: The housing 20 has a base 20a and two mutually opposite lateral housing parts 20b and supports the bracket 15, which is mounted rotatably via bearing pins 18, 18′ in corresponding bearings or through-openings 18a, as shown in FIG. 4. Thus, the bracket 15 is pivotable about a pivot axis A, which runs perpendicular to the longitudinal axis B. Between the lateral housing parts 20b, the swash plate 14 is arranged, which is enclosed by a steering ring 19 along its circumference. The swash plate 14 is preferably mounted rotatably in the steering ring 19, for example as shown in FIG. 11, by means of a roller bearing, or there a ball bearing, so that the swash plate 14 can be rotated with the main shaft 38, while the steering ring 19 does not rotate about the longitudinal axis B.

The steering ring 19 is spanned by the bracket 15 and is held in a through-opening 22 of the bracket 15 by means of a bearing ring 21 so as to be rotatable about an axis of rotation D perpendicular to the pivot axis A. For this purpose, the steering ring 19 has an axle stub 33.

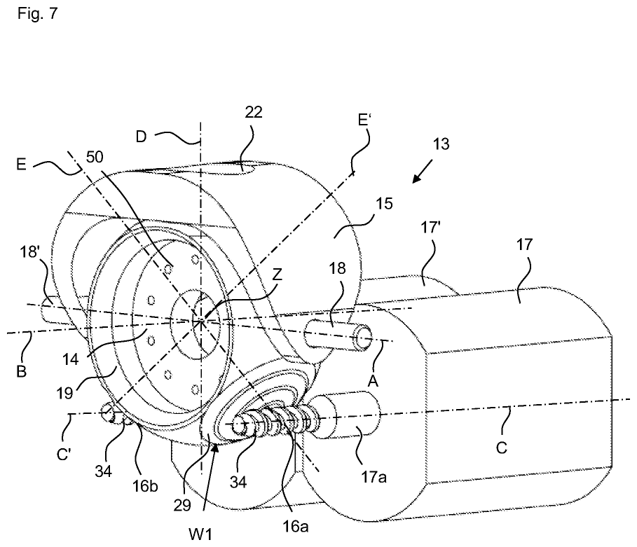

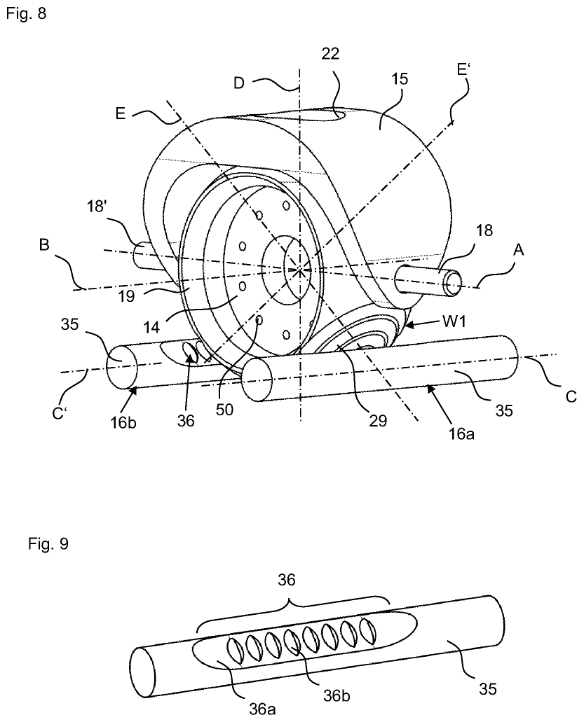

Below the base 20a in FIGS. 2 to 6, two motors 17, 17′ are arranged, of which the drive axes C, C′ are parallel to each other and are perpendicular both to the pivot axis A and to the longitudinal axis B, without intersecting the longitudinal axis B. Furthermore, in the illustrated view of the steering gear 13, the drive axes C, C′ are in a neutral starting position parallel to the axis of rotation D of the steering ring 19, which can however change depending on the displacement of the swash plate 14. In the example of FIGS. 7 and 8, the drive axes C, C′ also run parallel to each other, but also parallel to the longitudinal axis B and perpendicular to the pivot axis A, but without intersecting the pivot axis A. The motors 17, 17′ have drive shafts 17a, 17b, which are shown in FIGS. 3 to 7. The drive shafts 17a, 17b of the motors 17, 17′ protrude through through-openings with in each case two opening portions 30, 31 in the housing base 20a, as can be seen in particular in FIG. 4.

In FIGS. 3 to 7, force transmitters 16a,16b are arranged on the drive shafts 17a, 17b and transmit the rotational movement of the drive shafts 17a, 17b to the steering ring 19 for the purpose of carrying out a rotational/tilting movement. Each force transmitter 16a, 16b is coupled to the steering ring 19, preferably in a lower region of the steering ring 19 (shown in the figures) that forms a respective active portion W1, W2. The direct force transmission between force transmitter 16a, 16b and steering ring 19 takes place in the active portion W1, W2. The outer surface of the steering ring 19, the shape of which corresponds to a spherical layer or spherical disk, forms a spherical zone, wherein the two parallel cutting planes, imagined on a corresponding sphere, are equidistant or symmetrical to the diameter plane. The steering ring 19 thus has a partial spherical shape in the active portion W1, W2, in order to coordinate its movements, and, in the event of tilting, to maintain contact with the force transmitters 16a, 16b, by which the movements are initiated. The center point of the sphere, on which the spherical zone is based which represents the outer surface of the steering ring 19, is identical to an intersection point of the axis of rotation D of the steering ring 19 and the pivot axis A of the bracket 15, this intersection point defining the gimbal center Z of the gimbal suspension.

In contrast to the examples shown, the steering ring can also have, only in the active portions W1, E2, an outer surface formed as a spherical cap, of which the associated sphere segment is based on the same sphere center point, which is at the same time the gimbal center Z.

Various embodiments of force transmitters 16a, 16b and active portions W1, W2 of the steering ring 19 are described below; as shown in FIGS. 3 to 9, wherein the force transmitters 16a, 16b can be rotationally mounted drive cones, shown in FIGS. 3 to 6 and 10 to 13, or rotationally driven worm shafts, shown in FIG. 7, or also linearly driven drive rods, shown in FIGS. 8 and 9.

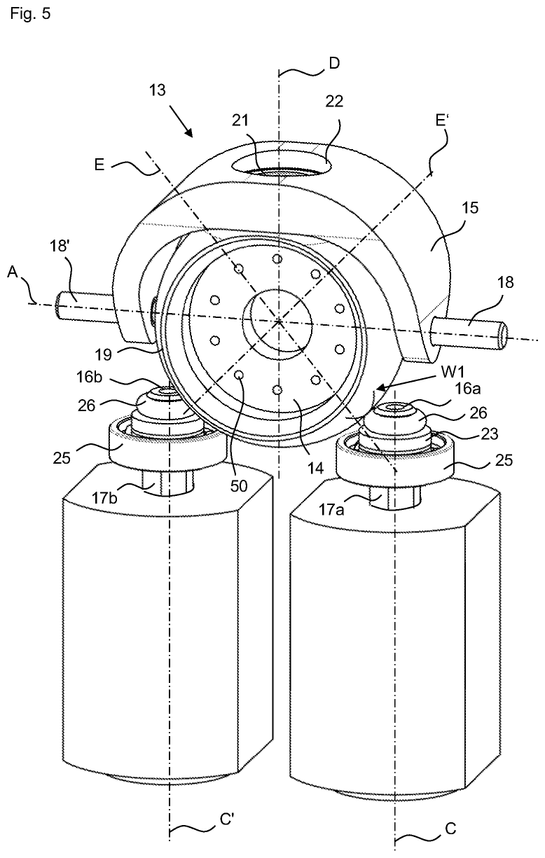

In FIGS. 3 and 4, the force transmitters 16a, 16b are shown with a functional form for illustrating the basic geometric principle. The force transmitters 16a, 16b are rotationally mounted drive cones with a cone head 23 and a cone shaft 42. Cone heads 23 are understood in the present case not only as cone heads with a conical or frustoconical shape, but also heads 23 with a spherical segment or spherical disk shape or a semi-ellipsoid shape, which offers a good ratio between the surfaces of cone head 23 and steering ring 19, wherein the respective contact planes on the surfaces of cone head 23 and steering ring 19 at their respective contact point K1, K2 are perpendicular to the axis E, E′ between this contact point K1, K2 and the gimbal center Z. The shape of the cone head 23 can be determined according to the type of operative connection (frictional, form-fit) between steering ring 19 and cone head 23. A cone head 23 with a spherical segment or spherical disk shape with a small radius in comparison to the radius of the steering ring 19 may be preferred, for example, for a frictional operative connection, as is described in connection with FIG. 5.

The movement transmission and the swash plate movement will be explained using the example of the embodiment in FIG. 3:

At the contact points K1, K2 between force transmitter 16a, 16b and steering ring 19, with FIG. 3 showing only contact point KI of the first force transmitter 16a with the steering ring 19, the rotational movement of the force transmitter 16a, 16b, when viewed infinitesimally at this point, is always parallel to the main axis B of the surgical instrument 1 and directed forward (distally) or rearward (proximally) with respect to the main axis B, depending on the direction of rotation. This movement is transmitted to the steering ring 19. On account of its gimbal suspension about the gimbal center Z, the steering ring 19 thus tilts about a tilt axis E, E′, which is assigned to the respective drive (motor 17, 17′).

If the first drive shaft 17a is now rotated by means of the first motor 17, and with it the first force transmitter 16a, the rotational movement of the first force transmitter 16a is transmitted to the first active portion W1 on account of the operative connection between the steering ring 19 and the first force transmitter 16a. The second force transmitter 16b remains stationary during this time, so that the steering ring 19, which is moved in the active portion W1 by the first force transmitter 16a, remains at the contact point K2 of the active portion W2 with the second force transmitter 16b. The steering ring 19 is thus tilted, specifically about the second tilt axis E′, which passes through the contact point K2 in the active portion W2 and the gimbal center Z and lies at an angle of 45° to both the axis of rotation D and the axis of rotation A. In this case, when the first force transmitter 16a turns counterclockwise, as viewed from above, and the second force transmitter 16b is stationary, in FIG. 3 a left-hand upper region of the steering ring 19 tilts rearward about the second tilt axis E′ and at the same time a right-hand lower region of the steering ring 19 tilts forward about the second tilt axis E′. Conversely, a rotation of the second force transmitter 16b at the same time as the first force transmitter 16b is stationary causes the steering ring 19 to tilt about the first tilt axis E, which, analogously to the second tilt axis E′, is at an angle of 45° to both the axis of rotation D and the axis of rotation A and accordingly passes through the contact point K1 of the first force transmitter 16a and the active portion W1 of the steering ring 19 and through the gimbal center Z, where the two tilt axes E, E′ intersect perpendicularly. If both force transmitters 16a, 16b turn, all the rotation or tilting of the steering ring 19 and thus of the swash plate 14 can be effected spatially.

This principle is also the basis for all other embodiments of force transmitters 16a, 16b shown here

Around the cone shaft 42 of each drive cone there is arranged, adjacent to the cone head 23, a bearing ring 25 (see FIGS. 2 to 6), which preferably houses a rolling bearing, for example a roller or ball bearing. The bearing ring 25 ensures guided rotation of the drive cones and thus good force transmission of the rotational movement to the steering ring 19. In order to hold the respective bearing ring 25, the housing 20 has, in its base 20a, two through-openings for the drive shafts 17a, 17b, each with two opening portions 30, 31, as can be seen in particular in FIG. 4. The opening portions 30, 31 are connected to each other and have a different diameter, wherein the opening portions 31, which are directed to their respective motor 17, 17′, have a smaller diameter than the overlying opening portions 30, which are directed to the steering ring 19. In this way, a shoulder 32 is formed at the connection point of both opening portions 30, 31, wherein each of the bearing rings 25 rests on the respective shoulder 32, as is shown in FIG. 4.

In FIG. 5, the force transmitters 16a, 16b are, as in FIGS. 3 and 4, rotationally mounted drive cones with a cone head 23, which is designed for a frictional operative connection to a friction element. The friction element shown is a toroidal friction band 26 circumferentially applied around the cone head 23, for example a rubber band or rubber ring, so that the operative connection between force transmitters 16a, 16b and steering ring 19 is produced here by means of frictional force. Alternatively, the friction element can be a friction-increasing material applied circumferentially to the cone head 23. For this purpose, but also for an improved hold of a circumferentially applied friction band 26 by means of a positive fit, a groove can be provided which surrounds the cone head 23 and into which the friction-increasing material penetrates during application, or a correspondingly shaped inner circumferential portion of the friction band 26 is received. The material used for the friction element preferably provides, with the steering ring, a frictional force higher than the frictional force which exists between the material of the cone head 23 and the steering ring 19. On account of the high frictional force between the cone head 23 and the active portion W1, W2 of the steering ring 19, the operative connection is provided by friction.

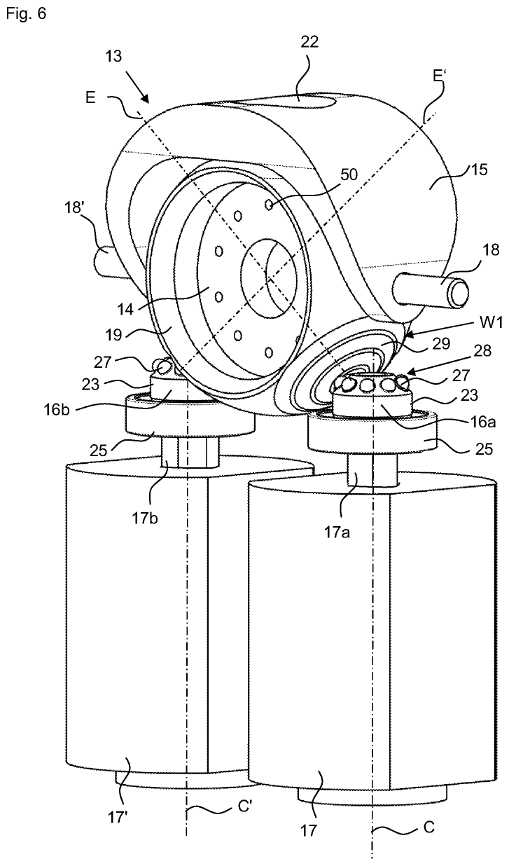

In FIG. 6, the operative connection between force transmitters 16a, 16b and steering ring 19 is provided by a form-fit engagement. Once again, the first and second force transmitters 16a, 16b are rotationally mounted drive cones with a cone head 23. In this embodiment, a plurality of drive knobs 27 are arranged circumferentially on the respective cone head 23 and form a circumferential knob ring 28. To ensure that the knobs 27 can engage with the steering ring 19, the steering ring 19 has, on its active portion W1 and correspondingly also on the second active portion (not visible in FIG. 6), recesses 29 which mesh with the drive knobs 27 of the two knob rings 28. In order to achieve transmission of movement, the recesses 29 are formed as concentric rings having a diameter adapted to the size and the tooth spacing of the knobs 27 and having a corresponding width in the active portion W1.

In FIG. 7, the operative connection between force transmitters 16a, 16b and steering ring 19 is likewise provided by a form-fit engagement, wherein the first and second force transmitters 16a, 16b are worm shafts 34. These worm shafts 34 run parallel to the longitudinal axis B, so that the drive axes C, C′ of the motors 17, 17′ also run parallel to the main axis B. As is already the case in the embodiment in FIG. 6, the steering ring 19 has, on its active portion W1 and correspondingly also on the second active portion (not visible in FIG. 7), concentrically arranged recesses 29 which mesh with the worm shaft 34 when the worm shaft 34 rotates. The movement transmission is also direct and corresponds to the movement transmission sequence already explained with reference to the rotationally driven drive cones.

FIGS. 8 and 9 show an alternative drive type: Instead of a rotary drive element, the first and second force transmitters 16a, 16b are toothed rods 35. These are equipped with a toothing portion 36. Analogously to the preceding embodiments, the steering ring 19 here has, on its active portion W1 and correspondingly also on the second active portion (not visible in FIG. 8), concentrically arranged recesses 29 which can mesh with the toothing portions 36 of the two toothed rods 35. The operative connection formed here is designed for a linear drive whose drive element (not shown in the figures), which in a steering gear according to the disclosure is intended to correspond to the drive shaft which defines the drive axis C. C′, does not rotate but is instead moved forward and back parallel to the main axis B. The toothing portion consists of an elongate recess 36a and of teeth 36a which are worked and arranged in this recess and which are linearly and equidistantly spaced from each other.

FIGS. 10 and 11 show an alternative gimbal bearing of the steering ring 19: The steering ring 19 comprises the swash plate 14, which is arranged on a universal joint plate 37, the latter being mounted on the main shaft 38. For this purpose, the swash plate 14 is mounted pivotably on a universal joint plate 37 via two bearing pins 40 offset 180° from each other, i.e. coaxially and diametrically arranged bearing pins 40, and the universal joint plate 37 is in turn mounted pivotably on the main shaft 38 via two bearing pins 39 offset 180° from each other, i.e. coaxially and diametrically arranged bearing pins 39. For better clarity, only one bearing pin 39 and one bearing pin 40 are shown in FIG. 11. The bearing pins 40 of the swash plate 14 and the bearing pins 39 of the universal joint plate 37 are arranged offset 90° from each other, wherein the pin axes define the gimbal center Z. This support makes it possible to pivot the swash plate 14 about two axes perpendicular to each other relative to the longitudinal axis B of the shaft 2, as a result of which the tool tip 6 (see FIG. 1) can be pivoted distally in all spatial directions relative to the longitudinal axis B of the shaft 2 via the steering wires 12. A bracket, as was provided in FIGS. 2 to 8, is not necessary in this internal gimbal bearing of the swash plate 14. To ensure that the steering ring 19 in this internal gimbal bearing can also rotate exclusively about the transverse axis and vertical axis, including superpositions, and its position in space is fixed and a rotation about the main axis B is prevented, the steering ring 19 in this case is coupled to the housing 20 in a rotationally fixed manner with respect to the longitudinal axis B by means of a pin (not shown), which extends radially outward from the underside of the steering ring 19, being guided in a groove 24 of the housing 20 (see FIG. 11), which groove 24 extends parallel to the longitudinal axis B along the radius of movement of the pin.

At its distal end 3, which corresponds to the proximal end 3 of the shaft 2, the main shaft 38 is surrounded by a protective cap 45, which protects the entry of the shaft 2 and thus of the main shaft 38 into the housing 20. The protective cap 45 is screwed with an outer thread 47 into a housing opening with a corresponding thread 48. At the distal end, the protective cap 45 further envelops a ball bearing 49, in which the main shaft 38 is rotatably mounted. Adjacent to this ball bearing 49, the guide element 44 is arranged around the main shaft 38 in order to allow the steering wires 12 to exit the shaft 2 and feed them to the swash plate 14, wherein the diameter of the bundle of steering wires widens accordingly.

Proximally behind the guide element 44, the steering wires 12 widening continuously with respect to the longitudinal axis B of the shaft 2 are fed to the swash plate 14. To secure the steering wires 12 on the swash plate 14, through-openings 50 for each steering wire 12 are formed in the swash plate 14, wherein, in the example shown, the steering wires 12 within the through-holes 50 are frictionally connected and affixed to the swash plate 14 by way of grub screws 41.

At its proximal end 46, the main shaft 38 is rotatably mounted in a further housing part 51 in a through-opening 52, for example by means of a plain bearing or a rolling bearing. Further proximally from the housing part 51, the actuation unit 4 can have a drive (not shown) for the movement of the actuation element 8.

The steering ring 19, which is used in the steering gear 13 according to FIGS. 10 and 11, does not require separate suspension by means of a bracket, since the gimbal suspension and bearing is effected on the main shaft 38. So that the swash plate 14 can rotate with the main shaft 38 about the longitudinal axis B, the swash plate 14 is rotatably mounted in the steering ring 19 by a rolling bearing. In FIG. 11, a ball can be seen as a rolling body 54, wherein the rolling bearing is preferably designed as a full spherical bearing. In the example shown, the bearing running surfaces are furthermore not formed in separate bearing shells, but directly on the outer surface of the swash plate 14 and on the inner surface of the steering ring 19, which is why the steering ring 19 has a filling nozzle 53 for filling the rolling element balls 54. The absence of bearing shells allows a more compact design of the swash plate/steering ring combination. After the rolling bearing is filled, the filling nozzle 53 is closed, for example with a screw as a stopper.

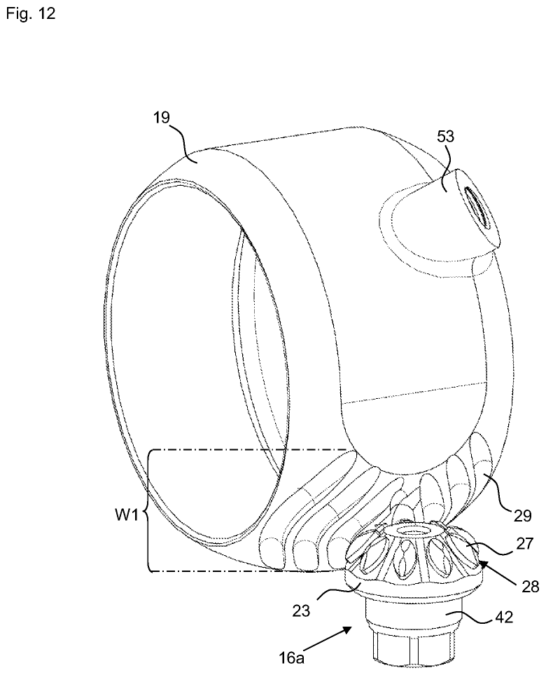

The steering ring 19 is moved with a rotationally driven drive cone as force transmitter 16a, whose cone head 23, as also shown in particular in FIGS. 11 and 12, protrudes through the through-opening in the housing base 20a and is guided in its rotation and supported by the bearing ring 25. The cone head 23 here has a knob ring 28 which carries semi-ellipsoid knobs 27. In FIGS. 11 and 12, the cone head 23, which rests on the cone shaft 42, is in engagement with the active portion W1 of the steering ring 19 and meshes, with its knob ring 28, with the active portion W1 of the steering ring 19. The active portion W1 of the steering ring 19 has arc-shaped recesses 29, which follow the ellipsoid shape of the knobs 27 of the cone head 23.

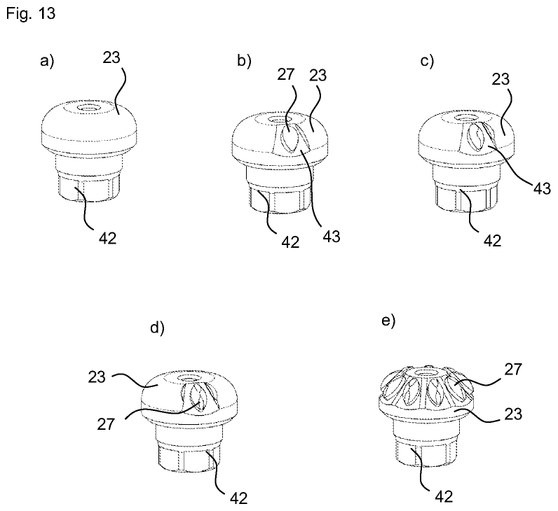

FIG. 13 a) to e) shows the steps involved in the machining of the cone head 23 shown in FIGS. 11 and 12. A first step according to FIG. 13 a) starts with a mushroom-shaped drive cone with a semi-ellipsoid cone head 23 on the shaft 42. A recess 43 is introduced into the envelope of the cone head 23, and an ellipsoid knob 27 is left standing (FIG. 13 b). In two further steps according to FIG. 13 c) and FIG. 13 d), the knobs 27 are brought into their semi-ellipsoid shape and faceted. The remaining knobs 27 are then created in the same way. FIG. 13 e) shows the final state when all the knobs 27 are formed in the semi-ellipsoid shape and the knob ring 28 is completely formed. Alternatively, and preferably for larger batch numbers, a drive cone shaped in this way can be produced by injection molding.

A surgical instrument 1 designed as described above is characterized in that a large number of thin steering wires 12 can be used to control the pivotable tool tip 6, and this control, based on the motor-driven steering gear 13 for the swash plate 14 on which the steering wires 12 are mounted, is sensitive, accurate and reproducible.

The drawings, the description and the claims contain numerous features in combination. A person skilled in the art will advantageously also consider the features on an individual basis and combine them to form further advantageous combinations. The present disclosure relates to a steering gear 13 for a surgical instrument 1, wherein the steering gear 13 can be arranged at the proximal end 3 of a shaft 2 and has a longitudinal axis B and a deflection mechanism 9 at the distal end 5. The steering gear 13 has two motorized drives and spatially orients a swash plate 14 by way of the adjustment angles of the two drives in order to control the deflection mechanism 9 of the surgical instrument 1. The swash plate 14 is arranged in a steering ring 19, wherein each of the two motorized drives has a drive shaft 17a, 17b driven by a motor 17, 17′, which drive shafts are directly in operative connection with the steering ring 19 via a force transmitter 16a, 16b. The first force transmitter 16a contacts the steering ring 19 on a first active portion W1, and the second force transmitter 16b contacts the steering ring 19 on a second active portion W2, wherein the steering ring 19 is mounted cardanically around a gimbal center Z and, at least on the first and second active portion W1, W2, is formed as sphere segment, wherein the active portions W1, W2 have a common sphere center at which a first tilt axis E, which is defined by a contact point K1 of the first force transmitter 16a with the steering ring 19 and by the gimbal center Z, and a second tilt axis E′, which is defined by a contact point K2 of the second force transmitter 16b with the steering ring 19 and by the gimbal center Z, intersect at right angles. Furthermore, a surgical instrument 1 is disclosed which comprises such a steering gear 13.

1. A steering gear for a surgical instrument, wherein the steering gear can be arranged at the proximal end of a shaft, which defines a longitudinal axis and has a deflection mechanism at the distal end, wherein the steering gear is configured to orient a swash plate disposed on a steering ring, the steering gear comprises:
two motorized drives configured to spatially orient the swash plate by way of an adjustment angle of the two motorized drives, the swash plate is designed to control the distal deflection mechanism of the surgical instrument, wherein;
the first of the two motorized drives has a first drive shaft, which is driven by a first motor and which is directly in operative connection with the steering ring via a first force transmitter, the first force transmitter directly contacting the steering ring on a first active portion, wherein the first force transmitter is arranged on the first drive shaft, which defines a first drive axis, and
the second of the two motorized drives has a second drive shaft, which is driven by a second motor and which is directly in operative connection with the steering ring via a second force transmitter, the second force transmitter directly contacting the steering ring on a second active portion, wherein the second force transmitter is arranged on the second drive shaft, which defines a second drive axis, and
wherein the steering ring is mounted cardanically around a gimbal center and, at least on the first and second active portions, has a spherical segment shape, wherein the spherical segment-shaped active portions have a common sphere center, which corresponds to the gimbal center, in which a first tilt axis, which is defined by a contact point of the first force transmitter with the steering ring and by the gimbal center, and a second tilt axis, which is defined by a contact point of the second force transmitter with the steering ring and by the gimbal center, intersect at right angles.

2. The steering gear as set forth in claim 1, wherein the cardanic mounting of the steering ring is provided by the fact that:
(a) the steering ring is suspended cardanically on a first fastening device, the first fastening device being a bracket which
is arranged on a portion of the steering ring facing away from the active portions and
at both ends is mounted on a housing rotatably about a pivot axis which runs perpendicular to the longitudinal axis and perpendicular to the drive axes and
has a receiving opening at its center,
wherein the steering ring is mounted in the receiving opening rotatably about an axis of rotation perpendicular to the pivot axis; or by the fact that:
(b) the steering ring mounted cardanically on a main shaft, extending coaxially to the longitudinal axis of the shaft, via the swash plate, which is mounted rotatably in the steering ring about the longitudinal axis or is firmly connected to the steering ring, wherein the swash plate is mounted pivotably on a universal joint plate via two bearing pins offset by 180° from each other, and the universal joint plate is mounted pivotably on the main shaft via two bearing pins offset by 180° from each other, and wherein the bearing pins of the swash plate and of the universal joint plate are offset by 90° from each other.

3. The steering gear as set forth in claim 1, wherein the operative connection provides a form-fit engagement since;
the first and second force transmitters are rotationally mounted drive cones having a cone head comprising circumferentially a plurality of drive knobs forming a knob ring, and wherein the steering ring has, on each active portion, recesses which mesh with the drive knobs of the two knob rings, or
the first and second force transmitters are worm shafts which run parallel to the longitudinal axis, and wherein the steering ring has, on each active portion, concentrically arranged recesses which mesh with the worm shaft.

4. The steering gear as set forth in claim 1, wherein the drive knobs have a hemispherical or semi-ellipsoid shape, and/or
the recesses are rings arranged concentrically one inside another or arc-shaped grooves.

5. The steering gear as set forth in claim 1, wherein the operative connection is provided by friction, since
the first and second force transmitters are rotationally mounted drive cones having a cone head, which comprises circumferentially a friction element, preferably a material which is applied around the cone head and which provides a higher friction than the material from which the cone head is made, or a friction band which is applied circumferentially around the cone head and which is a rubber band or a band with a roughened surface or a band with teeth or knobs.

6. The steering gear as set forth in claim 1, wherein the operative connection of the first force transmitter to the steering ring provides a form-fit engagement, and the operative connection of the second force transmitter to the steering ring is provided by frictional engagement,
wherein the form-fit engagement of the first force transmitter with the steering ring is provided by the fact that the first force transmitter is
a rotationally mounted drive cone with a cone head, which circumferentially comprises a plurality of drive knobs forming a knob ring, wherein the steering ring has, on the first active portion, recesses which mesh with the drive knobs of the two knob rings, or
a worm shaft which extends parallel to the longitudinal axis, and wherein the steering ring has, on the first active portion, recesses which are concentrically arranged and which mesh with the worm shaft,
and wherein the frictional engagement of the second force transmitter with the steering ring is provided by the fact that
the second force transmitter a rotationally mounted drive cone with a cone head, which includes circumferentially a friction element which is preferably
a material which is applied around the cone head and which provides a higher friction than the material from which the cone head is made, or
a friction band which is applied circumferentially around the cone head and which is a rubber band or a band with a roughened surface or a band with teeth or knobs.

7. The steering gear as set forth in claim 3, wherein the rotationally mounted drive cones each have a cone shaft around which a bearing ring is arranged adjacent to the cone head, and in that
the housing has a base with a through-opening for each drive axle, wherein each through-opening has a first opening portion with a diameter corresponding to a diameter of the bearing ring, and a second opening portion, coaxial to the first opening portion, with a diameter smaller than the diameter of the first opening portion, such that the housing forms, in each through-opening, a shoulder on which the respective bearing ring rests.

8. The steering gear as set forth in claim 1, wherein the operative connection
forms a linear drive, wherein the first and second force transmitters are toothed rods with a toothing portion, and wherein the steering ring has, on its active portion, concentrically arranged recesses which mesh with the toothed portions of the two toothed rods, or
is a cohesively bonded operative connection, wherein a first tension means is fastened with both ends to the first active portion, and the first tension means is wrapped around the first force transmitter, and a second tension means is fastened with both ends to the second active portion, and the second tension means is wrapped around the second force transmitter.

9. A surgical instrument comprising:
a shaft;
an actuation unit arranged at the proximal end of the shaft; and
a tool arranged at the distal end of the shaft with a tool tip which can be bent at an angle by means of a distal deflection mechanism and which can be controlled by a swash plate that can be spatially oriented by means of two drives, wherein the surgical instrument has the steering gear as set forth in claim 1, the steering gear configured to adjust the spatial orientation of the swash plate.

10. The surgical instrument as claimed in claim 9, characterized in that the actuation element is mounted axially displaceably in the shaft and is operatively connected at the proximal end to the actuation unit; and/or
the distal deflection mechanism of the bendable tool tip consists of pivot members which are arranged at the distal end of the shaft and which are connected to the steering gear by means of steering wires which run in the longitudinal direction of the shaft and are fastened to the swash plate.
